(19) United States
(12) Patent Application Publication
(10) Pub. No.: US 2006/0148776 A1
(43) Pub. Date: Jul. 6, 2006

(54) Drug formulations having long and medium chain triglycerides

(75) Inventors: Edqar H. Ulm, Encinitas, CA (US); Robert K. Mansfield, Carlsbad, CA (US); Marcus F. Boehm, San Diego, CA (US)

Correspondence Address: WILSON SONSINI GOODRICH & ROSATI, 650 PAGE MILL ROAD, PALO ALTO, CA, 94304-1050, US

(73) Assignee: Conforma Therapeutics Corporation, San Diego, CA (US)

(21) Appl. No.: 10/548,968

(22) PCT Filed: Oct. 4, 2003
(86) PCT No.: PCT/US03/31667

§ 371 Date: Jan. 18, 2006

Related U.S. Application Data
(60) Provisional application No. 60/454,812, filed on Mar. 13, 2003.
(60) Provisional application No. 60/478,430, filed on Jun. 12, 2003.
(60) Provisional application No. 60/491,050, filed on Jul. 29, 2003.

(30) Foreign Application Priority Data
Apr. 4, 2003 (US) US03/10533

(51) Int. Cl. A61K 31/685; A61K 31/33

(52) U.S. Cl. 514/183; 514/78; 514/547

(57) ABSTRACT

Drug formulations having emulsifying agents and both medium and long chain triglycerides are described. In preferred embodiments, the long chain triglycerides negate or lessen deleterious central nervous system effects that are caused by medium chain triglycerides.

### RELATED APPLICATIONS

This application is related to, claims priority to and incorporates by reference in their entireties each of Ulm et al., U.S. Provisional Patent Application Ser. No. 60/491,050, filed Jul. 29, 2003 and entitled ANSAMYCIN FORMULATIONS AND METHODS FOR PRODUCING AND USING SAME; Ulm et al., U.S. Provisional Patent Application Ser. No. 60/478,430, filed Jun. 12, 2003 and entitled PHOSPHOLIPID-BASED FORMULATIONS AND METHODS FOR PRODUCING AND USING SAME; Ulm et al., U.S. Provisional Patent Application Ser. No. 60/454,812, filed Mar. 13, 2003 and entitled HSP90-INHIBITOR FORMULATIONS AND DATA; and Ulm et al., PCT Patent Application Serial Number PCT/US03/10533, entitled NOVEL ANSAMYCIN FORMULATIONS AND METHODS FOR PRODUCING AND USING SAME, filed Apr. 4, 2003, which claims priority to U.S. Provisional Application Ser. No. 60/371,668, filed Apr. 10, 2002, and is entitled the same.

### FIELD OF INVENTION

The invention relates in general to pharmaceutical formulations and methods, and in more specific embodiments to emulsified formulations of ansamycins, e.g., 17-AAG.

### BACKGROUND

The following description includes information that may be useful in understanding the present invention. It is not an admission that any of the information provided herein is prior art or relevant to the presently claimed inventions, or that any publication specifically or implicitly referenced is prior art.

17-allylamino-geldanamycin (17-AAG) is a synthetic analog of geldanamycin (GDM). Both molecules belong to a broad class of antibiotic molecules known as ansamycins. GDM, as first isolated from the microorganism Streptomyces hygroscopicus, was originally identified as a potent inhibitor of certain kinases, and was later shown to act by stimulating kinase degradation, specifically by targeting “molecular chaperones,” e.g., heat shock protein 90s (HSP90s). Subsequently, various other ansamycins have demonstrated more or less such activity, with 17-AAG being among the most promising and the subject of intensive clinical studies currently being conducted by the National Cancer Institute (NCI). See, e.g., Federal Register, 66(129): 35443-35444; Erlichman et al., Proc. AACR (2001), 42, abstract 4474.

HSP90s are ubiquitous chaperone proteins that are involved in folding, activation and assembly of a wide range of proteins, including key proteins involved in signal transduction, cell cycle control and transcriptional regulation. Researchers have reported that HSP90 chaperone proteins are associated with important signaling proteins, such as steroid hormone receptors and protein kinases, including, e.g., Raf-1, EGFR, v-Src family kinases, Cdk4, and ErbB-2 ( Buchner J., 1999, TIBS, 24:136-141; Stepanova, L. et al., 1996, Genes Dev. 10:1491-502; Dai, K. et al., 1996, J. Biol. Chem. 271:22030-4). Studies further indicate that certain co-chaperones, e.g., Hsp70, p60/Hop/Sti1, Hip, Bag1, HSP40/Hdj2/Hsj1, immunophilins, p23, and p50, may assist HSP90 in its function (see, e.g., Caplan, A., Trends in Cell Biol., 9: 262-68 (1999).

Ansamycin antibiotics, e.g., herbimycin A (HA), geldanamycin (GM), and 17-AAG are thought to exert their anticancerous effects by tight binding of the N-terminus ATP-binding pocket of HSP90 (Stebbins, C. et al., 1997, Cell, 89:239-250). This pocket is highly conserved and has weak homology to the ATP-binding site of DNA gyrase (Stebbins, C. et al., supra; Grenert, J. P. et al., 1997, J. Biol. Chem., 272:23843-50). Further, ATP and ADP have both been shown to bind this pocket with low affinity and to have weak ATPase activity (Proromou, C. et al., 1997, Cell, 90: 65-75; Panaretou, B. et al., 1998, EMBO J., 17: 4829-36). In vitro and in vivo studies have demonstrated that occupancy of this N-terminal pocket by ansamycins and other HSP90 inhibitors alters HSP90 function and inhibits protein folding. At high concentrations, ansamycins and other HSP90 inhibitors have been shown to prevent binding of protein substrates to HSP90 (Scheibel, T., H. et al., 1999, Proc. Natl. Acad. Sci. U S A 96:1297-302; Schulte, T. W. et al., 1995, J. Biol. Chem. 270:24585-8; Whitesell, L., et al., 1994, Proc. Natl. Acad. Sci. U S A 91:8324-8328). Ansamycins have also been demonstrated to inhibit the ATP-dependent release of chaperone-associated protein substrates (Schneider, C., L. et al., 1996, Proc. Natl. Acad. Sci. U S A, 93:14536-41; Sepp-Lorenzino et al., 1995, J. Biol. Chem. 270:16580-16587). In either event, the substrates are degraded by a ubiquitin-dependent process in the proteasome (Schneider, C., L., supra; Sepp-Lorenzino, L., et al., 1995, J. Biol. Chem., 270:16580-16587; Whitesell, L. et al., 1994, Proc. Natl. Acad. Sci. USA, 91: 8324-8328).

This substrate destabilization occurs in tumor and non-transformed cells alike and has been shown to be especially effective on a subset of signaling regulators, e.g., Raf (Schulte, T. W. et al., 1997, Biocheenz. Biopliys. Res. Commnun. 239:655-9; Schulte, T. W., et al., 1995, J. Biol. Chem. 270:24585-8), nuclear steroid receptors (Segnitz, B., and U. Gehring. 1997, J. Biol. Chetn. 272:18694-18701; Smith, D. F. et al., 1995, Mol. Cell. Biol. 15:6804-12 ), v-src (Whitesell, L., et al., 1994, Proc. Natl. Acad. Sci. U S A 91:8324-8328) and certain transmembrane tyrosine kinases (Sepp-Lorenzino, L. et al., 1995, J. Biol. Chem. 270:16580-16587) such as EGF receptor (EGFR) and Her2/Neu (Hartmann, F., et al., 1997, Int. J. Cancer 70:221-9; Miller, P. et al., 1994, Cancer Res. 54:2724-2730; Mimnaugh, E. G., et al., 1996, J. Biol. Chem. 271:22796-801; Schnur, R. et al., 1995, J. Med. Chem. 38:3806-3812), CDK4, and mutant p53. Erlichman et al., Proc. AACR (2001), 42, abstract 4474. The ansamycin-induced loss of these proteins leads to the selective disruption of certain regulatory pathways and results in growth arrest at specific phases of the cell cycle (Muise-Heimericks, R. C. et al., 1998, J Biol. Chem. 273:29864-72), and apoptsosis, and/or differentiation of cells so treated (Vasilevskaya, A. et al., 1999, Cancer Res., 59:3935-40).

Recently, Nicchitta et al., WO 01/72779 (PCT/US01/09512), demonstrated that HSP90 can assume a different conformation upon heat shock and/or binding by the fluorophore bis-ANS. Specifically, Nicchitta et al. demonstrated that this induced conformation exhibits a higher affinity for certain HSP90 ligands than for a different form of HSP90 that predominates in normal cells. Commonly-owned application PCT/US02/39993 carries this discovery even further by demonstrating the utility and uses of cancer cell lystates as excellent sources of high affinity HSP90.

In addition to anti-cancer and antitumorgenic activity, HSP90 inhibitors have also been implicated in a wide variety of other utilities, including use as anti-inflammation agents, anti-infectious disease agents, agents for treating autoimmunity, agents for treating stroke, ischemia, cardiac disorders and agents useful in promoting nerve regeneration (See, e.g., Rosen et al., WO 02/09696 (PCT/US01/23640); Degranco et al., WO 99/51223 (PCT/US99/07242); Gold, U.S. Pat. No. 6,210,974 B1; DeFranco et al., U.S. Pat. No. 6,174,875). Overlapping somewhat with the above, there are reports in the literature that fibrogenetic disorders including but not limited to scleroderma, polymyositis, systemic lupus, rheumatoid arthritis, liver cirrhosis, keloid formation, interstitial nephritis, and pulmonary fibrosis also may be treatable. (Strehlow, WO 02/02123; PCT/US01/20578). Still further HSP90 modulation, modulators and uses thereof are reported in PCT/US03/04283, PCT/US02/35938, PCT/US02/16287, PCT/US02/06518, PCT/US98/09805, PCT/US00/09512, PCT/US01/09512, PCT/US01/23640, PCT/US01/46303, PCT/US01/46304, PCT/US02/06518, PCT/US02/29715, PCT/US02/35069, PCT/US02/35938, PCT/US02/39993, 60/293,246, 60/371,668, 60/331,893, 60/335,391, 06/128,593, 60/337,919, 60/340,762, and 60/359,484.

At present, ansamycins like many other lipophilic drugs are difficult to prepare for pharmaceutical applications, especially injectable intravenous formulations. To date, attempts have been made to use lipid vesicles and oil-in-water type emulsions, but these have thus far required complicated processing steps, harsh or clinically unacceptable solvents, and/or resulted in formulation instability. See generally Vemuri, S. and Rhodes, C. T., Preparation and characterization of liposomes as therapeutic delivery systems: a review, Pharmaceutica Acta Helvetiae 70, pp. 95-111(1995); see also PCT/US99/30631, published Jun. 29, 2000 as WO 00/37050. Commonly-owned application PCT/US03/10533, to which this application claims priority, describes emulsion formulations having medium chain triglycerides. However, medium chain fatty acids and triglycerides bearing such can lead to metabolic formation of octanoate, which can lead to undesired central nervous system effects such as somnolence, nausea, drowsiness and changes in EEG. See Cotter et al., Am. J. Clin. Nutr. 50:794-800 (1989); Miles et al., Journal of Parenteral and Enteral Nutrition 15:37-41 (1991); Traul et al., Food Chem. Toxicol. 38:79-98 (2000). To date, such negative effects have been off-set somewhat by the use of long chain fatty acids in the form of nutritional supplements, which compete with greater binding efficiency for a key octanoate pathway enzyme. However, to Applicants' knowledge to date they have not been combined with medium chain triglycerides and ansamycins.

A need therefore exists for alternative formulations and formulation methods that are relatively simple to manufacture and that ameliorate one or more of the foregoing deficiencies that typically accompany medium chain triglycerides.

### SUMMARY OF THE INVENTION

Applicants' presently claimed formulations are observed to provide for better tolerated intravenous administration of lipophilic compounds such as ansamycins by formulating such compounds together with long chain triglycerides as a component of the formulation.

In a first aspect, the invention features a pharmaceutical composition comprising a pharmacologically active compound, e.g., an ansamycin such as 17-AAG, in combination with an emulsifying agent (e.g., phospholipids such as found in lecithin) and oil. The oil may and preferably does contain long chain triglycerides. The composition can also contain medium chain triglycerides. The emulsifying agent and oil together constitute a lipid phase.

In some embodiments, the lipid phase constitutes 5-30% by weight of the total formulation, more preferably 5-20%.

In some embodiments, the overall w/w percent of long chain triglycerides does not exceed 10%, more preferably ranges at 7% or below, and more preferably still ranges at 6% or below to comport with viscosity constraints.

In some embodiments, medium chain triglycerides are present in a w/w ratio of from 10:0.0001 to 0.0001:10, and more preferably 10:1 to 1:10 relative to long chain triglycerides.

In some embodiments, the phospholipids are present in a range of from 3-10% w/w of the total.

In some embodiments, the triglycerides constitute 5-20% w/w of the total.

In some embodiments, triglycerides are present, at least in part, in the form of naturally existing oils, e.g., plant oils such as soy, sesame, safflower and corn.

In some embodiments, the composition further comprises one or more of water, a preservative (e.g., sodium edentate), cryoprotectant, buffer, chelating agent, and tonicifier.

In some embodiments, 1 7-AAG (1 7-allylaminogeldanamycin 1 7-allylamino-17-demethoxy-geldanamycin) is the drug and is present in an amount of 0.5 mg/ml to 4 mg/ml or 0.05% w/w to 0.4% w/w relative to the total formulation weight.

In one embodiment, the composition has the following components: 2 mg/ml 17-AAG, 3.3% soy oil, 6.6% lecithin, 9.9% Miglyol 812N, 7.5% sucrose, and water.

In another embodiment, the composition has the following components: 2 mg/ml 17-AAG, 7.5% lecithin, 15% Miglyol 812N, 10% sucrose, and water.

17-AAG is 17-allylaminogeldanamycin and has structure:

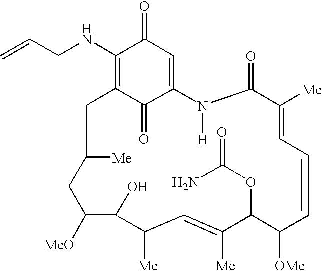

In some embodiments, the composition of the invention also comprises small chain triglyerides.

In one particularly preferred embodiment, the composition comprises long chain triglycerides in an amount sufficient to lessen or negate the incidence of medium chain triglyceride-mediated central nervous system (CNS) effects, especially if medium chain triglycerides are also present in the same composition. CNS effects are typically negative, undesirable effects, and include but are not limited to one or more of somnolence, nausea, drowsiness, and changes in EEG. In some embodiments, however, such effect(s) may be desirable in a given context, such that the relative amount of medium chain triglycerides is increased relative to long chain triglycerides.

In some embodiments, the composition is frozen and/or lyophilized, e.g., as generally described in PCT/US03/10533. In lyophilization embodiments, the weight percents of the individual triglycerides, phospholipids, drug, and non-volatile components necessarily increases over and above the ranges described above to comport with their relative fractional increase upon loss of water and any other volatile agents that may initially be present and lost thereafter upon lyophilization.

In some embodiments, the compositions are also formulated and/or stored in an inert atmospheric conditions, e.g., in the case of the latter a dark and/or light impervious bottle, vial, or ampoule.

Combinations of any of the foregoing embodiments are also contemplated where appropriate.

In another aspect the invention features a method of lessening the incidence of medium chain triglyceride-mediated central nervous system effects in a patient comprising providing a drug formulation comprising a pharmacologically active drug and long chain triglycerides, said long chain triglycerides present in an amount sufficient to reduce or negate the incidence of medium-chain fatty acid mediated central nervous system effects, and administering the product of step a) to a patient. Embodiments for this aspect may track any of the composition embodiments for the foregoing aspect and combinations thereof.

In another aspect, the invention features methods of using the pharmaceutical compositions, formulations, methods and products described above for treating or preventing a disorder in an organism, e.g., a mammal, by administering to the organism a pharmaceutically effective amount of product. The disorder, at least in the instance of mammalian treatment, is preferably selected from the group of disorders consisting of ischemia, proliferative disorders and neural damage, and includes an HSP90 inhibitor, e.g, one or more ansamycins, as the pharmacologically active drug. Proliferative disorders include but are not limited to tumors and cancers, inflammatory diseases, fungal infections, yeast infections, and viral infections. In some preferred embodiments, the mammal is human. In some preferred embodiments, the administration mode is intravenous, although as described in more detail, below, other modes of administration are also contemplated.

Advantages of the invention include, depending on the specific embodiment, one or more of ease of manufacture, the use of clinically acceptable reagents (e.g., having reduced environmental and/or patient toxicity), enhanced formulation stability, uncomplicated shipment and warehousing, simple pharmacy and bed-side handling, IV and systemic tolerance upon administration, and the negation of certain undesirable side-effects that often accompany medium chain fatty acids and triglyceride loads in the body. Other advantages, aspects, and embodiments will be apparent from the figures, the detailed description, and claims to follow.

### BRIEF DESCRIPTION OF THE DRAWINGS

FIG. 1 illustrates reduced somnolence in rats attributable to inclusion of long chain fatty acids in formulations that contain medium chain triglycerides.

### DESCRIPTION OF THE PREFERRED EMBODIMENTS

The formulations of the invention have particular merit in rendering water-insolubledrugs suitable for intravenous and other types of administration to a patient. The method of formulation is relatively simple, typically utilizes clinically acceptable reagents, and results in a product that affords storage, stability, and biotolerability advantages over existing methods and products.

### DEFINITIONS

The following claim terms have the following meanings, and claim terms not specifically appearing below have their common customary meaning as used in the art:

The term “pharmacologically active compound” is synonymous with “drug” and means any compound that exerts, directly or indirectly, a biological effect, in vitro or in vivo when administered to cultured cells or to an organism. The drug is preferably capable of encasement in liposomes and/or emulsification, and will typically, although not necessarily, be lipophilic.

The terms “evaporating” and “lyophilizing” do not necessarily imply 100% elimination of solvent and solution, and may entail lesser percentages of removal. In lyophilized embodiments, however, substantial removal is preferred, preferably about 95% or more.

An “inert atmospheric condition” is one that is relatively less reactive than the air of standard atmospheric conditions. The use of pure or substantially pure nitrogen gas during formulation is one example of such an inert atmospheric condition. Persons of ordinary skill in the art are familiar with others.

The term “hydrating” or rehydrating” means adding an aqueous solution, e.g., water or a physiologically compatible buffer such as Hanks's solution, Ringer's solution, or physiological saline buffer.

The term “about” is meant to embrace deviations of 20% from what is stated. The term “inclusive” when used in conjunction with the term “between” or “between about” means including the endpoints of the stated range.

The term “ansamycin” is a broad term which characterizes compounds having an “ansa” structure which comprises any one of benzoquinone, benzohydroquinone, naphthoquinone or naphthohydroquinone moities bridged by a long chain. Compounds of the naphthoquinone or naphthohydroquinone class are exemplified by the clinically important agents rifampicin and rifamycin, respectively. Compounds of the benzoquinone class are exemplified by geldanamycin (including its synthetic derivatives 17-allylamino-17-demothoxygeldanamycin (17-AAG) and 17-N,N-dimethylamino-ethylamino-17-demethoxygeldanamycin (DMAG)), dihydrogeldanamycin and herbamycin. The benzohydroquinone class is exemplified by macbecin. Ansamycins and benzoquinone ansamycins according to this invention. Ansamycins and benzoquinone ansamycins according to the invention may be synthetic, naturally-occurring, or a combination of the two, i.e., “semi-synthetic”, and may include dimers and conjugated variant and prodrug froms. Some exemplary benzoquinone ansamycins useful in the processes of the invention and their methods of preparation include but are not limited to those described, e.g., in U.S. Pat. No. 3,595,955 (describing the preparation of geldanamycin), U.S. Pat. Nos. 4,261,989, 5,387,584, and 5,932,566. Geldanamycin is also commercially available, e.g., from CN Biosciences, an Affiliate of Merck KGaA, Darmstadt, Germany, headquartered in San Diego, Calif., USA (cat. no. 345805). The biochemical purification of the geldanamycin derivative, 4,5-Dihydrogeldanamycin and its hydroquinone from cultures of Streptomyces hygroscopicus (ATCC 55256) are described in International Application Number PCT/US92/10189, assigned to Pfizer Inc., published as WO 93/14215 on Jul. 22, 1993, and listing Cullen et al. as inventors; an alternative method of synthesis for 4,5-Dihydrogeldanamycin by catalytic hydrogenation of geldanamycin is also known. See e.g., Progress in the Chemistry of Organic Natural Products, Chemistry of the Ansanzycin Antibiotics, 33:278 (1976). Other ansamycins that can be used in connection with various embodiments of the invention are described in the literature cited in the “Background” section, above.

“Oils” include fatty acids and glycerides containing the same, e.g., mono-, di- and triglycerides as known in the art. The fatty acids and glycerides for use in the invention can be saturated and/or unsaturated, natural and/or synthetic, charged or neutral. “Synthetic” may be entirely synthetic or semisynthetic as those terms are known in the art. The oils may also be homogenous or heterogeneous in their constituents and/or origin.

“Medium chain triglycerides” as used herein are triglyceride compositions having fatty acids ranging in size from 8-12 linear carbon atoms in length, and more preferably 8-10 carbon atoms in length. Various embodiments of the invention include the use of Miglyol® 812, which is offered by CONDEA (Cranford, N.J., USA). Miglyol® 812 contains roughly 50-65% Caprylic acid (8 carbons) and 30-45% Capric acid (10 carbons). Caproic acid (6 carbon atoms) is also present, up to a maximum of about 2%, as is Lauric Acid (12 carbons). Present in still a lesser amount (1% max) is Myristic acid (14 carbons). Condea also offers Miglyol® 810, 818, 829, and 840, and it is anticipated that one or more of these other Miglyol® solutions, as well as other medium chain triglyceride solutions can also be used more or less successfully in connection with various aspects and embodiments of the invention. As to the latter, one of ordinary skill in the art knows their identity, source and/or manner of preparation, and can acquire or prepare them without undue investigation or experimentation. Miglyol 812N has monographs in the European Pharmacopeia as Medium Chain Triglycerides, the British Pharmacopeia as Fractionated Coconut Oil, and the Japanese Pharmacopeia as Caprylic/Capric Triglycerides. Other sources of medium chain triglycerides include coconut oil, palm kernel oil and butter.

“Short chain triglycerides” are triglyceride compositions having fatty acids less than 8 linear carbon atoms in length.

“Long chain triglycerides” are triglyceride compositions having fatty acids greater than 12 linear carbon atoms in length. A common source of these is vegetable oil, for example, soy oil, which typically contains 55-60% linoleic acid (9,12-octadecadienoic acid), 22% oleic acid (cis-9-octadecenoic acid), and lesser amounts of palmitic and stearic acid.

The terms “short, “medium” and “long” can also be used with respect to a fatty acid alone, in which case the definitions of such include, respectively, less than 8 linear carbon atoms, 8 to 12 linear carbon atoms, and greater than 12 linear carbon atoms.

“Emulsifying agents” are synonymous with “surfactants” and include but are not limited to phospholipids such as lecithins. “Lecithins” are naturally occurring mixtures of diglycerides of stearic, palmitic, and oleic acids, linked to the choline ester of phosphoric acid. The term surfactant or emulsifying agent also embraces phosphatidylcholine, which distinct compound is well known. Preferred emulsifying agents for use with the invention are soya lecithin, e.g., Phospholipon 90G as supplied by American Lecithin Company (Oxford, Conn., USA). Phospholipon 90G has previously been used in parenteral nutritional products such as Intralipid® at a concentration of about 1.2%, Doxil® (doxorubicin) at about 1%, Ambisome® (amphotericin B) at about 2%, and Propofol® at about 1.2%. In the case of the latter, see, e.g., U.S. Pat. No. 6,140,374. The surfactant/emulsifying agent is typically present in a concentration of about 0.5-25% w/v based on the amount of the water and/or other components into which the surfactant is dissolved. Preferably, the surfactant is present in a concentration of about 0.5-10% w/v, most preferably about 1-8% w/v.

Examples of anionic surfactants include sodium lauryl sulfate, lauryl sulfate triethanolamine, sodium polyoxyethylene lauryl ether sulfate, sodium polyoxyethylene nonylphenyl ether sulfate, polyoxyethylene lauryl ether sulfate triethanolamine, sodium cocoylsarcosine, sodium N-cocoylmethyltaurine, sodium polyoxyethylene (coconut)alkyl ether sulfate, sodium diether hexylsulfosuccinate, sodium a-olefin sulfonate, sodium lauryl phosphate, sodium polyoxyethylene lauryl ether phosphate, perfluoroalkyl carboxylate salt (manufactured by Daikin Industries Ltd. under the trade name of UNIDINE DS-101 and 102).

Examples of cationic surfactants include dialkyl(C₁₂-C₂₂)dimethylammonium chloride, alkyl(coconut)dimethylbenzylammonium chloride, octadecylamine acetate salt, tetradecylamine acetate salt, tallow alkylpropylenediamine acetate salt, octadecyltrimethylammonium chloride, alkyl(tallow) trimethylammonium chloride, dodecyltrimethylammonium chloride, alkyl(coconut) trimethylammonium chloride, hexadecyltrimethylammonium chloride, biphenyltrimethylammonium chloride, alkyl(tallow)-imidazoline quaternary salt, tetradecylmethylbenzylammonium chloride, octadecyidimethylbenzylammonium chloride, dioleyidimethylammonium chloride, polyoxyethylene dodecylmonomethylammonium chloride, polyoxyethylene alkyl(C₁₂-C₂₂)benzylammonium chloride, polyoxyethylene laurylmonomethyl ammonium chloride, 1-hydroxyethyl-2-alkyl(tallow)-imidazoline quaternary salt, and a silicone cationic surfactant having a siloxane group as a hydrophobic group, a fluorine-containing cationic surfactant having a fluoroalkyl group as a hydrophobic group (manufactured by Daikin Industries Ltd. under the trade name of UNIDINE DS-202).

Examples of nonionic surfactants include polyoxyethylene lauryl ether, polyoxyethylene tridecyl ether, polyoxyethylene cetyl ether, polyoxyethylene polyoxypropylene cetyl ether, polyoxyethylene stearyl ether, polyoxyethylene oleyl ether, polyoxyethylene nonylphenyl ether, polyoxyethylene octylphenyl ether, polyoxyethylene monolaurate, polyoxyethylene monostearate, polyoxyethylene monooleate, sorbitan monolaurate, sorbitan monostearate, sorbitan monopalmitate, sorbitan monostearate, sorbitan monooleate, sorbitan sesquioleate, sorbitan trioleate, polyoxyethylene sorbitan monolaurate, polyoxyethylene sorbitan monopalmitate, polyoxyethylene sorbitan monostearate, polyoxyethylene sorbitan monooleate, polyoxyethylene polyoxypropylene block polymer, polyglycerin fatty acid ester, polyether-modified silicone oil (manufactured by Toray Dow Corning Silicone Co., Ltd. under the trade names of SH3746, SH3748, SH3749 and SH3771), perfluoroalkyl ethyleneoxide adduct (manufactured by Daikin Industries Ltd. under the trade names of UNIDINE DS-401 and DS-403), fluoroalkyl ethyleneoxide adduct (manufactured by Daikin Industries Ltd. under the trade name of UNIDINE DS-406), and perfluoroalkyl oligomer (manufactured by Daikin Industries Ltd. under the trade name of UNIDINE DS-451).

A “physiologically acceptable carrier” refers to a carrier or diluent that does not cause significant irritation to an organism and does not abrogate the biological activity and properties of the administered compound.

An “excipient” refers to a substance added to a pharmacological composition to further facilitate administration of a compound. Examples of excipients include but are not limited to calcium carbonate, calcium phosphate, various sugars and types of starch, cellulose and cellulose derivatives, gelatin, vegetable oils and polyethylene glycols. These can also be physiologically acceptable carriers, as described above, e.g., sucrose. Further falling within the definition of excipient are bulking agents. A “bulking agent” generally provides mechanical support for a lyophile formulation by allowing the dry formulation matrix to maintain its conformation. Preferred are sugars. Sugars as used herein include but are not limited to monosaccharides, disaccharides, oligosaccharides and polysaccharides. Specific examples include but are not limited to fructose, glucose, mannose, trehalose, sorbose, xylose, maltose, lactose, sucrose, dextrose, and dextran. Sugar also includes sugar alcohols, such as mannitol, sorbitol, inositol, dulcitol, xylitol and arabitol. Mixtures of sugars may also be used in accordance with this invention. Various bulking agents, e.g., glycerol, sugars, sugar alcohols, and mono and disaccharides may also serve the function of isotonizing agents, as described above. It is preferred that the bulking agents be chemically inert to drug(s) and have no or extremely limited detrimental side effects or toxicity under the conditions of use. In addition to bulking agent carriers, other carriers that may or may not serve the purpose of bulking agents include, e.g., adjuvants and diluents as well known and readily available in the art.

A preferred bulking agent for the invention is sucrose. Without being bound by theory, sucrose is thought to form an amorphous glass upon freezing and subsequent lyophilization, allowing for potential stability enhancement of the formulation by forming a dispersion of the oil droplets containing the active ingredient in a rigid glass. Stability may also be enhanced by virtue of the sugar acting as a replacement for the water lost upon lyophilization. The sugar molecules, rather than the water molecules, become bonded to the interfacial phospholipid through hydrogen bonds. Other bulking agents which possess these characteristics and which may be substituted include but are not limited to cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may also be added, e.g., cross-linked polyvinyl pyrrolidone, agar, or alginic acid or a salt thereof such as sodium alginate.

The claim phrase “an amount that lessens or negates the incidence of medium chain triglyceride-mediated central nervous system effects” can be empirically determined by one or ordinary skill without undue experimentation using the starting points described herein.

Emulsification

Emulsions comprising an oil phase and an aqueous phase are widely known in the art as carriers of therapeutically active ingredients or as sources of parenteral nutrition. Emulsions can exist as either oil-in-water or water-in-oil forms. If, as is the case in the current instance, the therapeutic ingredient is particularly soluble in the oil phase the oil-in-water type is the preferred embodiment. Simple emulsions are thermodynamically unstable systems from which the oil and aqueous phases separate (coalescence of oil droplets). Incorporation of emulsifying agent(s) into the emulsion is critical to reduce the process of coalescence to insignificant levels.

To prevent or minimize oxidative degradation or lipid peroxidation, antioxidants, e.g., alpha-tocopherol and butylated hydroxytoluene, and preservatives such as edentate may be included in addition to, or as an alternative to, oxygen deprivation (e.g., formulation in the presence of inert gases such as nitrogen and argon, and/or the use of light resistant containers).

Emulsification can be effected by a variety of well known techniques, e.g., mechanical mixing, homogenization (e.g., using a polytron or Gaulin high-energy-type instrument), vortexing, and sonication. Sonication can be effected using a bath-type or probe-type instrument. Microfluidizers are commercially available, e.g., from Microfluidics Corp., Newton, Mass., are further described in U.S. Pat. No. 4,533,254, and make use of pressure-assisted passage across narrow orifices. Pressure assisted extrusion through various commercially available polycarbonate membranes may also be employed. Low pressure devices also exist that can be used. These high and low pressure devices can be used to select for and/or modulate vesicle size.

Sterilization by filtration techniques. Filtration can include a pre-filtration through a larger diameter filter, e.g., a 0.45 micron filter, and then through smaller filter, e.g., a 0.2 micron filter. The preferred filter medium is cellulose acetate (Sartorius-Sartobran™).

Lyophilization

Lyophilization is the removal or substantial removal of liquid from a sample, e.g., by sublimation, and as described in the section above entitled “solvent removal.”

Characterization and Assessment of Chemical and Physical Stability

Phospholipids and degradation products may be determined after being extracted from emulsions. The lipid mixture can then be separated in a two-dimensional thin-layer chromatographic (TLC) system or in a high performance liquid chromatographic (HPLC) system. In TLC, spots corresponding to single constituents can be removed and assayed for phosphorus. Total phosphorous in a sample can be quantitatively determined, e.g., by a procedure using a spectrophotometer to measure the intensity of blue color developed at 825 nm against water. In HPLC, phosphatidylcholine (PC) and phosphotidylglycerol (PG) can be separated and quantified with accuracy and precision. Lipids can be detected in the region of 203-205 nm. Unsaturated fatty acids exhibit high absorbance while the saturated fatty acids exhibit lower absorbance in the 200 nm wavelength region of the UV spectrum. As an example, Vemuri and Rhodes, supra, described the separation of egg yolk PC and PG on Licrosorb Diol and Licrosorb S1-60. The separations used a mobile phase of acetonitrile-methanol with 1% hexane-water (74:16:10 v/v/v). In 8 minutes, separation of PG from PC was observed. Retention times were approximately 1.1 and 3.2 min, respectively.

Emulsion visual appearance, average droplet size, and size distribution can be important parameters to observe and maintain. There are a number of methods to evaluate these parameters. For example, dynamic light scattering and electron microscopy are two techniques that can be used. See, e.g., Szoka and Papahadjopoulos, Annu. Rev. Biophys. Bioeng., 9:467-508 (1980). Morphological characterization, in particular, can be accomplished using freeze fracture electron microscopy. Less powerful light microscopes can also be used.

Emulsion droplet size distribution can be determined, e.g., using a particle size distribution analyzer such as the CAPA-500 made by Horiba Limited (Ann Arbor, Mich., USA), a Coulter counter (Beckman Coulter Inc., Brea, Calif., USA), or a Zetasizer (Malvern Instruments, Southborough, Mass., USA).

Stability Determination Using HPLC

Similar to the methods described above for the lipid components of the emulsion, the chemical stability of the therapeutically active ingredient, e.g, 17-AAG, can be assessed by HPLC after extraction of the emulsion. Specific assay procedures can be developed that allow for the separation of the therapeutically active ansamycin from its degradation products. The extent of degradation can be assessed either from the decrease in signal in the HPLC peak associated with the therapeutically active ansamycins and/or by the increase in signal in the HPLC peak(s) associated with degradation products. Ansamycins, relative to other components of the emulsion components, are easily and quite specifically detected at their absorbance maximum of 345 nm.

Modes of Formulation and Administration

Although intravenous administration is preferred in various aspects and embodiments of the invention, one of ordinary skill will appreciate that the methods can be modified or readily adapted to accommodate other administration modes, e.g., oral, aerosol, parenteral, subcutaneous, intramuscular, intraperitoneal, rectal, vaginal, intratumoral, or peritumoral. The following discussion is largely known to the person of skill but is nevertheless provided as a backdrop to illustrate other possibilities for the invention. It will be appreciated that various of the commentary below overlaps with what has already been discussed above.

Pharmaceutical compositions may be manufactured utilizing conventional mixing, dissolving, granulating, dragee-making, levigating, emulsifying, encapsulating, entrapping or lyophilizing processes.

Pharmaceutically acceptable compositions may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Some excipients and their use in the preparation of formulations have already been described. Others are known in the art, e.g., as described in PCT/US99/3063 1, Remmington's Pharmaceutical Sciences, Meade Publishing Co., Easton, Pa. (most recent edition), and Goodman and Gilman's The Pharmaceutical Basis of Therapeutics, Pergamon Press, New York, N.Y. (most recent edition).

For injection, the agents may be formulated in aqueous solutions, preferably in physiologically compatible buffers such as Hanks's solution, Ringer's solution, or physiological saline buffer, as each are well-known in the art. For transmucosal administration, penetrants appropriate to the barrier to be permeated are used in the formulation. Such penetrants are generally known in the art.

Formulations of the invention, as described previously, and upon hydration of the lyophilized cakes, are well suited for immediate or near-immediate parenteral administration by injection, e.g., by bolus injection or continuous infusion. Formulations for injection may be presented in unit dosage form, e.g., in ampoules or in multi-dose containers, with an added preservative, e.g., edetate. As discussed previously, the pharmaceutical compositions of the invention can be stored in an inert environment, e.g., in ampoules or other packaging that are light-resistant or oxygen-free.

Dose Range

A phase I pharmacologic study of 17-AAG in adult patients with advanced solid tumors determined a maximum tolerated dose (MTD) of 40 mg/m² when administered daily by 1-hour infusion for 5 days every three weeks. Wilson et al., Am. Soc. Clin. Oncol., abstract, Phase I Pharmacologic Study of 17-(Allylamino)-17-Denzethoxygeldanamycin (AAG)in Adult Patients with Advanced Solid Tumors (2001). In this study, mean ± SD values for terminal half-life, clearance and steady-state volume were determined to be 2.5±0.5 hours, 41.0±13.5 L/hour, and 86.6±34.6 L/m². Plasma Cmax levels were determined to be 1860±660 nM and 3170±1310 nM at 40 and 56 mg/m2. Using these values as guidance, it is anticipated that the range of useful patient dosages for formulations of the present invention will include between about 0.40 mg/m² and 4000 mg/m² of active ingredient. M² represents surface area. Standard algorithms exist to convert mg/m² to mg drug/kg bodyweight.

The following examples are offered by way of illustration only, and components and steps included therein are not intended to be limiting of the invention unless specifically recited in the claims. Examples 1-5 and 9 are borrowed from commonly owned application PCT/US03/10533, entitled NOVEL ANSAMYCIN FORMULATIONS AND METHODS FOR PRODUCING AND USING SAME, filed Apr. 4, 2003, and to which this application claims priority.

### EXAMPLES

### Example 1

### Preparation of 17-AAG; Alternative 1

To 45.0 g (80.4 mmol) of geldanamycin in 1.45 L of dry THF in a dry 2 L flask was added drop-wise over 30 minutes, 36.0 mL (470 mmol) of allyl amine in 50 mL of dry THF. The reaction mixture was stirred at room temperature under nitrogen for 4 hr at which time TLC analysis indicated the reaction was complete [(GDM: bright yellow: Rf=0.40; (5% MeOH-95%CHCl3); 17-AAG: purple: Rf=0.42 (5% MeOH-95% CHCl3)]. The solvent was removed by rotary evaporation and the crude material was slurried in 420 mL of H2O:EtOH (90:10) at 25 ° C., filtered and dried at 45 ° C. for 8 hr to give 40.9 g (66.4 mmol) of 17purple crystals (82.6% yield, >98% pure by HPLC monitored at 254 nm). MP 206-212 ° C. as determined using differential scanning colorimetry (DSC). 1H NMR and HPLC are consistent with the desired product.

### Example 2

### Preparation of a Low Melting Point Form of 17-AAG

An alternative method of purification is to dissolve the crude 17-AAG from example 1 in 800 mL of 2-propyl alcohol (isopropanol) at 80° C. and then cool to room temperature. Filtration followed by drying at 45° C. for 8 hr gives 44.6 g (72.36 mmol) of 17-AAG as purple crystals (90% yield, >99% pure by HPLC monitored at 254 nm). MP 147-175° C. as determined using differential scanning colorimetry (DSC). 1H NMR and HPLC are consistent with the desired product.

### Example 3

### Preparation of a Low Melting Point Form of 17-AAG, Alternative 1

An alternative method of purification is to slurry the 17-AAG product from example 2 in 400 mL of H2O:EtOH (90:10) at 25° C., filtered and dried at 45° C. for 8 hr to give 42.4 g (68.6 mmol) of 17-AAG as purple crystals (95% yield, >99% pure by HPLC monitored at 254 nm). MP 147-175° C. 1H NMR and HPLC are consistent with the desired product.

### Example 4

### Preparation of a 17-AAG Emulsion

The 17-AAG obtained from any one of Examples 1-4, above, is dissolved in ethanol. The following Table illustrates a 4000 gm batch preparation of 17-AAG made according to one embodiment of the invention. The skilled artisan will recognize that the procedure can be scaled up or down, that variations can be made with respect to the amounts of individual components, etc., and that additional components not listed may also be added.

| | | Grams for |
|---|---|---|
| Component | % w/w | 4000 g batch |
| 17-AAG | 0.2 | 8 |
| Miglyol 812 | 15 | 600 |
| Phospholipon 90G | 7.5 | 300 |
| EDTA disodium, dihydrate, | 0.005 | 0.2 |
| USP | | |
| Sucrose, NF | 15 | 600 |
| Water-for-injection, USP | QS | QS |
| 0.2N NaOH | To adjust pH to 6.0 ± 0.2 | As needed |

17-AAG (CNF-101) is weighed in a 5L polypropylene beaker. Ethanol is added in an amount approximately 50× the drug weight and the solution sonicated in a water bath to disperse the drug. Miglyol 812 (Sasol North America Inc; Houston, Tex., USA) and Phospholipon 90G (American Lecithen Co., Oxford, Conn., USA) are then added to the dispersion and the mixture placed on a stir plate and stirred until the solids are more or less completely dissolved. A sonicator bath and/or heat to approximately 45° C. may be used to help dissolve the solids. The solution may be checked using an optical microscope to ensure desired dissolution.

A stream of dry air or nitrogen (National Formulary) gas is forced over the liquid surface in combination with vigorous stirring to evaporate the ethanol until the ethanol content is reduced, preferably to less than 50% of its initial presence, more preferably to less than 10%, and most preferably to about 5% or less, w/w. The solution can be checked under an optical microscope equipped with polarizing filters to ensure the desired level of dissolution, preferably complete dissolution (no crystals or precipitate).

EDTA (disodium, dihydrate, USP), sucrose, and water for injection (WFI) are weighed into a 5 L polypropylene beaker and stirred until the solids are dissolved. The aqueous phase is then added to the oil phase and thorough mixing effected using a high-speed emulsifier equipped with an emulsion head at 5000 RPM until the oil adhering to the surface is “stripped off.” Shearing rate is then increased to 10000 RPM for 2-5 minutes to obtain a uniform primary emulsion. Laser light scattering (LLS) may be used to measure the average droplet size, and the solution may further be checked, e.g., under an optical microscope to determine the relative presence or absence of crystals and solids.

The emulsion pH is adjusted to 6.0±0.2 with 0.2N NaOH. The primary emulsion is then passed through a Model 11OS microfluidizer (Microfluidics Inc., Newton, Mass., USA) operating at static pressure of about 110 psi (operating pressure of 60-95 psi) with a 75-micron emulsion interaction chamber (F20Y) for 6-8 passages until the average droplet size is ≦190 nm. LLS may be used following the individual passages to evaluate progress. The solution may further be checked for the presence of crystals using polarized light under an optical microscope.

In a laminar flow hood, the emulsion is then passed across a 0.45 micron Gelman mini capsule filter (Pall Corp., East Hills, N.Y., USA), and then across a sterile 0.2 micron Sartorius Sartobran P capsule filter (500 cm²) (Sartorius AG, Goettingen, Germany). Pressure up to 60 PSI is used to maintain a smooth and continuous flow. Filtrate is collected in one or more polypropylene bottles and immediately placed in a −20° C. freezer. A 1-ml aliquot may be set aside for testing using laser light scattering (LLS) and/or high performance liquid chromatography (HPLC).

### Example 5

### Alternative Preparation of 17-AAG Emulsion Formulation

When using ethanol to facilitate the dissolution of 17-AAG into the oil phase of the emulsion, it is most common to first dissolve 17-AAG in the ethanol using sonication followed by addition of the emulsifying agent and medium chain triglyceride to that solution. Sonication and stirring are then employed to effect solution of all the components.

Alternatively, 17-AAG can be brought into solution in the oil phase without ethanol being present by heating a preformed emulsifying agent in triglyceride solution, e.g., Phospholipon in Miglyol® 812, preferably to 65° C. or more, adding to this the drug, e.g., 17-AAG, and mixing, e.g., by stirring and/or sonication. It has also been discovered that a lower melting point form of 17-AAG from example 2 prepared through crystallization of 17-AAG from isopropanol rather than ethanol more readily can be dissolved into the Phospholipon in Miglyol solution at room temperature. See commonly-owned PCT/US01/29715, entitled “PROCESS FOR PREPARING . . . [17-AAG] AND OTHER ANSAMYCINS,” filed Sep. 18, 2002.

The products of both examples 5 and 6 result in a light purple, milky emulsion having a mean oil droplet size in the range of about 200 nm or less. The droplet size is stable at −20° C., 2-8° C., or room temperature for periods in excess of two months. Concentrations as high as 3 mg/ml have been achieved overall and as high as 20-30 mg/ml in the oil phase alone. When stored at 40° C., degradants of 17-AAG are seen after approximately 2 weeks.

The mixed-solvent solution of the drug is subjected to vacuum evaporation of the ethanol component resulting in a solution of 17-AAG in Miglyol. Emulsification can be accomplished by mechanical mixing, by treating with ultrasonic irradiation, and finally by passage through a microfluidizer, although it will be understood that the terms “emulsify” and “emulsification” should not be limited to such processing events and that other emulsification techniques exist and can be used alternatively or in tandem with one or more of the preceding techniques.

### Example 6

### Adding Long Chain Triglycerides

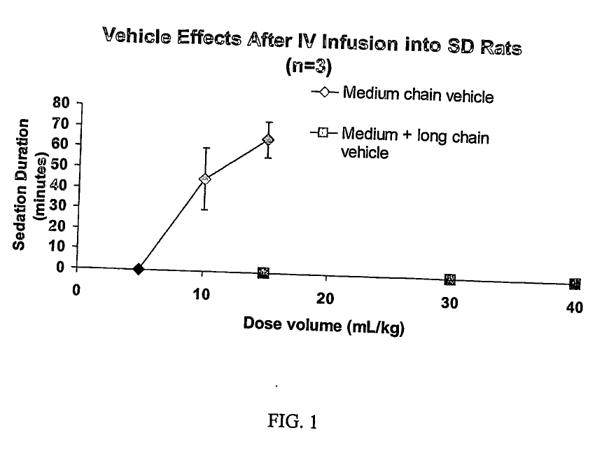

A variation of the processes described above includes the addition of long chain triglycerides, e.g., in the form of soya oil. As detailed in FIG. 1 for 17-AAG, a source of long chain triglycerides (soya oil) is mixed with Miglyol 812N (a source of medium chain triglycerides) and an emulsifying agent (Phospholipon 90G (PL90G)), in the following respective w/w proportions: 16.7%:50.0%:33.3%. This is homogenized until the PL90G is completely dissolved (˜20,000 rpm for about 20 minutes). To this is then added I% w/w 17-AAG, which is homogenized/dissolved in at ˜20,000 rpm for about 5 minutes. This constitutes the “oil phase.” This oil phase (1 part) is then slowly added to 3.6 parts of an aqueous phase (9.375% w/w sucrose, 0.0063% w/w EDTA in sterile water for injection) while homogenizing the latter at ˜12,000-15,000 rpm. The resulting mix is then pH'd to 6.0±0.2 using sodium hydroxide and/or hydrochloric acid as necessary. This “primary” emulsion is then microfluidized by passage through an F12Y interaction chamber and filter sterilized using a 0.2 μm polyethersulfone (PES) filter membrane.

Using the foregoing procedures, the following two formulations were made:

| Ingredients (w/w) | Formula 1 | Formula 2 |
|---|---|---|
| 17-AAG | 2 mg/ml | 2 mg/ml |
| Soy Oil | 3.3% | — |
| Phospholipon 90G | 6.6% | 7.5% |
| (Soy lecithin) | | |
| Miglyol 812N | 9.9% | 15% |
| Sucrose | 7.5% | 10% |
| Sodium Edetate | 0.005% | 0.005% |
| Ster. Water | 72.5% | 67.3% |

### Example 7

### Lyophilization

Lyophilization of the emulsions from Examples 5 and 6 may be accomplished according to a scheme similar to that in the following Table.

| Initial | Final | | |
|---|---|---|---|
| Temp. | Temp. | Pressure | |
| (° C.) | (° C.) | (mTorr) | Action |
| 25 | −40 | Ambient | Ramp at 1° C./min |
| −40 | −40 | Ambient | Hold for 60 min |
| −40 | −40 | 50 | Condenser at |
| | | | −60° C. to−80° C. |
| −40 | −28 | 50 | Ramp at 1° C./min |
| −28 | −28 | 50 | Hold for 7200 min |
| −28 | 30 | 50 | Ramp at 1° C./min |
| 30 | 30 | 50 | Hold for 300 min |

Complete
Stopper vials under N₂ at approximately 0.9 atm

The stability profile for the lyophilized 17-AAG emulsion was as follows when stored at 2-8° C., and following reconstitution:

Time (weeks)

| Test | 0 | 2 | 4 | 10 |
|---|---|---|---|---|
| Assay (% initial) | 100 | 98.5 | 97.7 | 97.0 |
| Purity (area %) | 98.8 | 97.2 | 97.8 | 97.8 |
| DropletSize (μm) | 0.187 | 0.200 | 0.197 | 0.190 |

### Example 8

### Long Chain Triglycerides Inhibit Somnolence

Miglyol 812N, when administered rapidly, can cause sedation due to the metabolic release of octanoate. During the intravenous infusion in rats of 17-AAG emulsion (Miglyol 812N oil) sedation was observed at infusion rates greater than 1.1 gm total lipid/Kg/hr. See FIG. 2. Sedation was also noted in dogs given intravenous iinfusions of the 17-AAG emulsion formulation at rates greater than 1.13 gm total lipid/kg/hr. To counter this, soybean oil was added as described above to compete with the metabolism of Miglyol 812N in-vivo to reduce octanoate fatty acid produced during intravenous infusions. In the soybean oil/Miglyol 812N CF237 emulsions, no sedation was observed acutely in rats at infusion rates of up to about 40 gm total lipid/kg/hr. Thus, the combination of soybean oil with Miglyol 812N greatly improves tolerability of the CF237 emulsion formulation with regard to sedation. Similarly, no sedation was observed in monkeys administered six doses of the CF237 emulsion formulation as an intravenous infusion of 12 mL formulation/kg/hr, and no vein irritation was observed.

### Example 9

### Preparation of Other Ansamycins for Similar Formulation

Ansmaycins other than 17-AAG

Essentially any ansamycin can be substituted for 17-AAG and formulated as described in the above examples. Various such ansamycins and their preparation are detailed in PCT/US03/04283. The preparation of two of these are described below.

Compound 563: 17-(benzoyl)-aminogeldanamycin. A solution of 17-aminogeldanamycin (1 mmol) in EtOAc was treated with Na₂S₂O₄ (0.1 M, 300 ml) at RT. After 2 h, the aqueous layer was extracted twice with EtOAc and the combined organic layers were dried over Na₂SO₄, concentrated under reduce pressure to give 18,21-dihydro-17-aminogeldanamycin as a yellow solid. This latter was dissolved in anhydrous THF and transferred via cannula to a mixture of benzoyl chloride (1.1 mmol) and MS4A Å (1.2 g). Two hours later, EtN(i-Pr)₂ (2.5 mmol) was further added to the reaction mixture. After overnight stirring, the reaction mixture was filtered and concentrated under reduce pressure. Water was then added to the residue which was extracted with EtOAc three times, the combined organic layers were dried over Na₂SO₄ and concentrated under reduce pressure to give the crude product which was purified by flash chromatography to give 17-(benzoyl)-aminogeldanamycin. Rf=0.50 in 80:15:5 CH2Cl2: EtOAc:MeOH. Mp=218-220° C. 1H NMR (CDC13) 0.94 (t, 6H), 1.70 (br s, 2H), 1.79 (br s, 4H), 2.03 (s, 3H), 2.56 (dd, 1H), 2.64 (dd, 1H), 2.76-2.79 (m, 1H), 3.33 (br s, 7H), 3.44-3.46 (m, 1H), 4.325 (d, 1H), 5.16 (s, 1H), 5.77 (d, 1H), 5.91 (t, 1H), 6.57 (t, 1H), 6.94 (d, 1H), 7.48 (s, 1H), 7.52 (t, 2H), 7.62 (t, 1H), 7.91 (d, 2H), 8.47 (s, 1H), 8.77 (s, 1H).

Compound 237: A dimer. 3,3′-diamino-dipropylamine (1.32 g, 9.1 mmol) was added dropwise to a solution of Geldanamycin (10 g, 17.83 mmol) in DMSO (200 ml) in a flame-dried flask under N₂ and stirred at room temperature. The reaction mixture was diluted with water after 12 hours. A precipitate was formed and filtered to give the crude product. The crude product was chromatographed by silica chromatography (5% CH₃OH/CH₂Cl₂) to afford the desired dimer as a purple solid. The pure purple product was obtained after flash chromatography (silica gel); yield: 93%; mp 165° C.; 1H NMR (CDCl3) 0.97 (d, J=6.6 Hz, 6H, 2CH3), 1.0 (d, J=6.6 Hz, 6H, 2CH3), 1.72 (m, 4 H, 2 CH2), 1.78 (m, 4 H, 2CH2), 1.80 (s, 6 H, 2 CH3), 1.85 (m, 2H, 2CH), 2.0 (s, 6H, 2CH3), 2.4 (dd, J=11 Hz, 2H, 2CH), 2.67 (d, J=15 Hz, 2H, 2CH), 2.63 (t, J=10 HZ, 2H, 2CH), 2.78 (t, J=6.5 Hz, 4H, 2CH2), 3.26 (s, 6H, 2OCH3), 3.38 (s, 6H, 20CH3), 3.40 (m, 2H, 2CH), 3.60 (m, 4H, 2CH2), 3.75 (m, 2H, 2CH), 4.60 ( d, J=10 Hz, 2H, 2CH), 4.65 (Bs, 2H, 20H), 4.80 (Bs, 4H, 2NH2), 5.19 (s, 2H, 2CH), 5.83 (t, J=15 Hz, 2H, 2CH═), 5.89 (d, J=10 Hz, 2H, 2CH═), 6.58 (t, J=15 Hz, 2H, 2CH═), 6.94 (d, J=10 Hz, 2H, 2CH═), 7.17 (m, 2H, 2NH ), 7.24 (s, 2H, 2CH═), 9.20 (s, 2H, 2NH); MS (m/z)1189 (M+H).

The corresponding HCl salt was prepared by the following method: an HCl solution in EtOH (5 ml, 0.123N) was added to a solution of compound #237 (1 gm as prepared above) in THF (15 ml) and EtOH (50 ml) at room temperature. The reaction mixture was stirred for 10 min. The salt was precipitated, filtered and washed with large amount of EtOH and dried in vacuo.

One of ordinary skill will appreciate that the parameters described in the preceding examples and tables can be adjusted depending on the conditions used, and depending on whether and to what extent the methods of formulation and amounts of materials used are scaled up or down, or varied, with respect to one another.

The foregoing examples are not limiting and merely representative of various aspects and embodiments of the present invention. All documents cited are indicative of the levels of skill in the art to which the invention pertains. The disclosure of each document cited is incorporated by reference herein to the same extent as if each had been incorporated by reference in its entirety individually, although none of the documents is admitted to be prior art. To the extent any of the definitions expressly stated herein conflict with any of those appearing in any of the art or priority documents cited herein, the definitions expressly stated herein control.

One skilled in the art will readily appreciate that the present invention is well adapted to carry out the objects and obtain the ends and advantages mentioned, as well as those inherent therein. The methods and compositions described illustrate preferred embodiments, are exemplary, and are not intended as limitations on the scope of the invention. Certain modifications and other uses will occur to those skilled in the art, and are encompassed within the spirit of the invention, as defined by the scope of the claims.

The reagents described herein are either commercially available, e.g., from Sigma-Aldrich, or else readily producible without undue experimentation using routine procedures known to those of ordinary skill in the art.

It will be readily apparent to one skilled in the art that varying substitutions and modifications may be made to the invention without departing from the scope and spirit of the invention. Thus, such additional embodiments are within the scope of the invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms, and each has a different meaning within the patent laws. The terms and expressions which have been employed are used as terms of description and not of limitation, and there is no intention in the use of such terms and expressions of excluding any equivalents of the features shown and described, or portions thereof. It is recognized that various modifications are possible within the scope of the invention claimed. Thus, it should be understood that although the present invention has been specifically disclosed by preferred embodiments, optional features, modifications and variations of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group, and exclusions by way of proviso of individual members as appropriate.

Other embodiments are within the following claims.

1. A pharmaceutical composition comprising:
an HSP90 inhibitor;
an emulsifying agent; and
an oil, said oil comprising both medium chain triglycerides and long chain triglycerides.

2. The pharmaceutical composition of claim 1 wherein said HSP90 inhibitor is an ansamycin.

3. The pharmaceutical composition of claim 2 wherein said ansamycin is geldanamycin or a geldanamycin derivative.

4. The pharmaceutical composition of claim 3 wherein said geldanamycin derivative is selected from 17-AAG and DMAG.

5. The pharmaceutical composition of claim 1 wherein said medium chain triglycerides are present in a w/w ratio of from 10:1 to 0.01:10 relative to said long chain triglycerides.

6. The pharmaceutical composition of claim 1 that is an oil in water emulsion having a lipid phase and an aqueous phase, and wherein said lipid phase comprises 5-30% by weight of the total.

7. The pharmaceutical composition of claim 1 wherein said phospholipids are present in the form of lecithin.

8. The pharmaceutical composition of claim 4 wherein lecithin is present in an amount of from 3-10% w/w of an oil in water emulsion.

9. The pharmaceutical composition of claim 1 wherein the composition is 3-10% w/w phospholipids and 5-20% w/w oil.

10. The pharmaceutical composition of claim 1 wherein the composition comprises no more than 10% w/w long chain triglycerides.

11. The pharmaceutical composition of claim 10 wherein the composition comprises less than or equal to 7% w/w of long chain triglycerides.

12. The pharmaceutical composition of claim 10 wherein said long chain triglycerides contain fatty acid esters comprised of 16-18 linear carbon units.

13. The pharmaceutical composition of claim 1 wherein said phospholipids are in the form of lecithin, optionally soy or egg lecithin.

14. The pharmaceutical composition of claim 4 wherein said ansamycin is a low melting point isoform of 17-AAG having a melting pointless than 200° C.

15. The pharmaceutical composition of claim 14 wherein said low melting point isoform is 17-AAG having a melting point of 147-175° C.

16. The pharmaceutical composition of claim 1 wherein said oil comprises one or more natural oils selected from the group consisting of soy, sesame, safflower, and corn.

17. The pharmaceutical composition of claim 1 that is an emulsion, optionally lyophilized.

18. The pharmaceutical composition of claim 1 wherein the composition comprises one or more of water, a preservative, cryoprotectant, buffer, chelating agent, and tonicifier.

19. The pharmaceutical composition of claim 1 comprising Miglyol 812N.

20. The pharmaceutical composition of claim 1 that comprises comprising 17-AAG in an amount of 0.5 mg/ml to 4 mg/ml.

21. The pharmaceutical composition of claim 1 that comprises comprising 17-AAG in the amount 0.05% w/w to 0.4% w/w.

22. The pharmaceutical composition o f claim 1 comprising the following ingredients: 2 mg/ml 17-AAG, 3.3% soy oil, 6.6% lecithin, 9.9% Miglyol 812N, 7.5% sucrose, and water.

23. The pharmaceutical composition of claim 1 comprising the following ingredients: 2 mg/ml 17-AAG, 7.5% lecithin, 15% Miglyol 812N, 10% sucrose, and water.

24. The pharmaceutical composition of claim 22 further comprising sodium edetate.

25. The pharmaceutical composition of claim 24 wherein said sodium edetate is present at 0.005% w/w.

26. The pharmaceutical composition of claim 1 wherein said long chain triglycerides are present in an amount that lessens or negates the incidence of medium chain triglyceride-mediated central nervous system effects.

27. The pharmaceutical composition of claim 26 wherein said central nervous system effects are selected from one or more of somnolence, nausea, drowsiness, and changes in EEG.

28. The pharmaceutical composition of claim 1 further comprising small chain triglycerides.

29. A method of lessening the incidence of medium chain triglyceride-mediated central nervous system effects in a patient receiving a drug formulation having medium-chain triglycerides as a component of said formulation, comprising:
(a) providing a drug formulation comprising an asamycin and both medium and long chain triglycerides, said long chain triglycerides present in an amount sufficient to reduce or negate the incidence of medium-chain fatty acid mediated central nervous system effects ; and
(b) administering the product of step (a) to a patient.

30. The method of claim 29 wherein said central nervous system effects are selected from one or more of somnolence, nausea, drowsiness, and changes in EEG.

31. The pharmaceutical composition of claim 1 wherein the composition is lyophilized, frozen, thawed, or reconstituted.

32. The pharmaceutical composition of claim 1 that is stored in an inert environment.

33. The pharmaceutical composition of claim 12 wherein said triglycerides are selected from the group consisting of linoleic acid, oleic acid, palmitic acid, stearic acid and combinations thereof.
